(19) United States
(12) Patent Application Publication
(10) Pub. No.: US 2026/0007603 A1
(43) Pub. Date: Jan. 8, 2026

(54) ALPHA-KETOGLUTARATE (AKG)-BASED POLYMERIC MICROPARTICLES AND METHODS OF USE

(71) Applicant: UNIVERSITY OF IOWA RESEARCH FOUNDATION, Iowa City, IA (US)

(72) Inventors: Hongli Sun, Iowa City, IA (US); Zhuozhi Wang, Iowa City, IA (US)

(73) Assignee: UNIVERSITY OF IOWA RESEARCH FOUNDATION, Iowa City, IA (US)

(21) Appl. No.: 19/261,537

(22) Filed: Jul. 7, 2025

Related U.S. Application Data
(60) Provisional application No. 63/668,654, filed on Jul. 8, 2024.

(51) Int. Cl. A61K 9/16; A61K 31/352; A61K 31/4965; C08G 65/48

(52) U.S. Cl. CPC A61K 9/1647; A61K 31/352; A61K 31/4965; C08G 65/485

(57) ABSTRACT

In certain embodiments, the present invention provides a biocompatible polymer comprising a backbone comprising one or more units of formula (I) in the backbone:

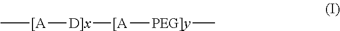

wherein A is Alpha-ketoglutarate (AKG); D is 1,8-octanediol (8diol) or 1,10-decanediol (10diol); PEG is polyethylene glycol; and the x: y ratio is between 100 to 200. In certain embodiments, the present invention also provides microparticle compositions and methods of use thereof.

### CROSS-REFERENCE TO RELATED APPLICATION

This application claims priority to U.S. Provisional Application No. 63/668,654 that was filed on Jul. 8, 2024. The entire content of the application referenced above is hereby incorporated by reference herein.

### FEDERAL GRANT SUPPORT

This invention was made with government support under DE029159 awarded by the National Institutes of Health. The government has certain rights in the invention.

### BACKGROUND

Annually, more than 600,000 patients have bone defects repaired in the U.S., at a cost greater than $2.5 billion. The prevalence of bone disease and the cost of repair are rising at an unprecedented rate, due primarily to the aging of the population. Aging not only reduces the body's ability to repair bone defects, but it is also a main factor of severe periodontitis with high chronic inflammation, which causes bone loss, tooth loss and osseointegration failure of dental implants. Current treatments for age-related bone loss, including antiresorptive bisphosphonate, denosumab, and anabolic human parathyroid hormone (PTH) approved for osteoporosis treatment, have significant adverse effects, e.g., osteonecrosis of the jaws (ONJ) and osteosarcoma.

Reconstruction of large bone defects caused by trauma, congenital anomalies, or pathology remains clinical challenge. Although autologous tissue remains the gold standard, its application is impeded by limited availability and donor site morbidity. Biomaterial-based tissue engineering presents an alternative strategy to address the challenges posed by autologous reconstruction. Biomaterial-mediated delivery of growth factors, such as bone morphogenetic protein 2 (BMP2), and transplantation of stem cells are an intensively studied approach for promoting bone regeneration. However, the low efficacy of BMP2 necessitates high doses, which can lead to serious side effects. Similarly, stem cells transplantation is fraught with challenges, including potential immunological rejection and the elevated risk of tumorigenesis. Therefore, a need exists to develop innovative biomaterials with robust pro-osteogenic properties, thereby reducing reliance on these intricate biological mediators.

### SUMMARY

Polyesters are used routinely by those skilled in the art in various drug delivery systems. Polyesters that degrade into useful biologically active compounds have now been developed. Accordingly, the invention provides a polymer of the invention, which is a polymer comprising a backbone, wherein the backbone comprises a diol conjugated to alpha-ketoglutarate and to polyethylene glycol. In certain embodiments, the backbone comprises one or more groups that will yield a biologically active compound upon hydrolysis of the polymer.

The present invention provides a biocompatible polymer comprising a backbone comprising one or more units of formula (I) in the backbone:

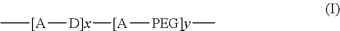

wherein
A is Alpha-ketoglutarate (AKG);
D is 1,8-octanediol (8diol) or 1,10-decanediol (10diol);
PEG is polyethylene glycol; and
x:y is between 100:1 to 200:1.

In the polymer of the invention, there are 3 moieties of the polymer backbone, which are A (AKG), D (diol) and PEG. A bonds with either D or PEG, but D does not bond with PEG. Thus, for the polymer -[A-D] x-[A-PEG]_{y}-, the ratio x:y represents the molar ratio of D and PEG in the polymer. The molar ratio of A in the polymer is 50% no matter what x or y is, and x:y is between 100:1 to 200:1 (i.e., when there is 100 to 200 units of D, there is only 1 unit of PEG). The invention also provides a polymeric microparticle (MP) composition comprising a first polymer, wherein the first polymer is the polymer the invention.

The invention also provides a pharmaceutical composition comprising the polymer of the invention or the MP composition of the invention, and a pharmaceutically acceptable carrier.

The invention also provides a method of increasing bone regeneration by administering to an animal in need of such therapy an effective amount of the polymer of the invention or the MP composition of the invention, or the pharmaceutical composition of the invention.

The invention also provides a method of increasing osteogenic differentiation by administering to an animal in need of such therapy an effective amount of the polymer of the invention or the MP composition of the invention, or the pharmaceutical composition of the invention.

The invention also provides a method of increasing intracellular phagocytosis by administering to an animal in need of such therapy an effective amount of the polymer of the invention or the MP composition of the invention, or the pharmaceutical composition of the invention.

The invention also provides a method of contacting pre-osteoblast MC3T3-E1 or primary bone marrow mesenchymal stem cells (MSCs) with the polymer of the invention or the MP composition of the invention to promote osteoblastic differentiation and/or mineralization.

The present invention provides in certain embodiments a kit comprising the conjugate described above, a container, and a package insert or label indicating the administration of the conjugate for treating bone loss.

### BRIEF DESCRIPTION OF THE DRAWINGS

FIGS. 1A-1C. AKG polymer (PAKG) synthesis, characterization and microparticles (MPs) fabrication, characterization. (FIG. 1A) Scheme of PAKG-10diol, PAKG-10diol-PEG, PAKG-8diol, PAKG-8diol-PEG synthesis. For PAKG-10diol and PAKG-8diol, x:y represents the molar ratio of D and PEG in the polymer, and x:y is between 100:1 to 200:1 (i.e., when there is 100 to 200 units of D, there is only 1 unit of PEG). In PEG-6K, x is about 136. (FIG. 1B) Schematic illustration of the fabrication of PAKG MPs. (FIG. 1C). Size distribution of PLLA, PLGA, PAKG-10diol, PAKG-10diol-PEG, PAKG-8diol, PAKG-8diol-PEG MPs (fabricated by homogenization) and their size distribution.

FIGS. 2A-2D. Rationale of AKG polymer design, selection (effect of carbon chain length of diol and PEG) and PAKG MPs-enhanced osteoblastic differentiation and mineralization. (FIG. 2A). ALP activity of MC3T3-E1 cultured with different doses of PAKG MPs for 7 days. (FIG. 2B) MC3T3-E1 cell proliferation with different concentrations of 1,8-octanediol and 1,10-decanediol. (FIG. 2C) Dose optimization of PAKG-8diol and PAKG-8diol-PEG MPs by ALP activity. (FIG. 2D) Mineralization of MC3T3-E1 with PAKG-8diol and PAKG-8diol-PEG MPs was studied. Alizarin Red S quantitation after staining at 4 weeks.

FIGS. 3A-3C. Effect of microparticle size on osteoblastic differentiation and mineralization. (FIG. 3A) Size of PAKG-8diol-PEG(S), PAKG-8diol(S) and PAKG-8diol-PEG(S) MPs. (FIG. 3B) ALP activity of MC3T3-E1 and mBMSCs with PLLA(S), PLGA(S), PAKG-8diol-PEG (L), PAKG-8diol(S), PAKG-8diol-PEG(S) MPs. (FIG. 3C) MC3T3-E1 cultured with PAKG-8diol-PEG (L), PAKG-8diol(S), PAKG-8diol-PEG(S) MPs for 24 days and mBMSCs cultured with PLLA(S), PLGA(S), PAKG-8diol-PEG(S) MPs for 28 days after Alizarin Red S staining. Alizarin Red S quantification after staining. Data are expressed as mean±SD (n=3, *p<0.05, **p<0.01, ***p<0.001, ****p<0.0001).

FIGS. 4A-4B. Osteoblast cell internalization of PAKG MPs through phagocytosis. (FIG. 4A) MC3T3-E1 internalization of PAKG-8diol-PEG MPs with Cytochalasin D (CCD, a phagocytosis inhibitor) and Pitstop 2 (a clathrin-independent endocytosis inhibitor) (Scale bars=50 μm) and quantitation of relative internalized MPs (right). (FIG. 4B) ALP activity of DAPI and Phalloidin MC3T3-E1 cultured with PAKG MPs, and PAKG MPs in the presence of CCD (50 nM) or Pitstop 2 (30 μM).

FIGS. 5A-5D. PAKG enables PLGA MPs for intracellular drug delivery. (FIG. 5A) Zeta-potential of various MPs. (FIG. 5B) Release profile of phenamil from PLGA and PLGA-PAKG MPs for 4 weeks. Upper line is PLGA-PAKG-Phe, lower line is PLGA-Phe. (FIG. 5C) ALP activity of MC3T3-E1 on day7. (FIG. 5D) Alizarin Red S quantification after MC3T3-E1 cultured with different MPs or Phe treatment in OC medium for 2 weeks. scale bars=500 μm. Data are expressed as mean±SD (n=3, *p<0.05, **p<0.01, ***p<0.001, ****p<0.0001).

FIG. 6. PAKG MPs promote mouse cranial bone regeneration. Quantification measurement of total new bone volume (BV) and the ratio of bone volume vs total volume in defected area (BV/TV) in different treatment groups. All the results were expressed as means±SD (n=3˜5, *p<0.05, **p<0.01).

### DETAILED DESCRIPTION

Polymers are used routinely by those skilled in the art in various delivery systems. The biocompatible polyesters of the invention are useful in a variety of applications where delivery of a biologically active compound is desired. Examples of such applications include, but are not limited to, medical, dental and cosmetic uses.

The polymers of the invention may be prepared in accordance with methods commonly employed in the field of synthetic polymers to produce a variety of useful products with valuable physical and chemical properties. The polymers can be readily processed into pastes or solvent cast to yield films, coatings, microspheres and fibers with different geometric shapes for design of various medical implants, and may also be processed by compression molding and extrusion.

Medical implant applications include the use of polyesters to form shaped articles such as vascular grafts and stents, bone plates, sutures, implantable sensors, implantable drug delivery devices, stents for tissue regeneration, and other articles that decompose into non-toxic components within a known time period.

Polymers of the present invention can also be incorporated into oral formulations and into products such as skin moisturizers, cleansers, pads, plasters, lotions, creams, gels, ointments, solutions, shampoos, tanning products and lipsticks for topical application.

Accordingly, the invention provides a polymer of the invention, which is a polymer comprising a backbone, wherein the backbone comprises a diol conjugated to alpha-ketoglutarate and to polyethylene glycol.

### Polymers

The present invention provides a biocompatible polymer comprising a backbone comprising one or more units of formula (I) in the backbone:

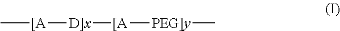

wherein
A is Alpha-ketoglutarate (AKG);
D is 1,8-octanediol (8diol) or 1,10-decanediol (10diol);
PEG is polyethylene glycol; and
the ratio x:y is between 100:1 to 200:1.

In certain aspects, the biocompatible polymer comprises-AKG-8diol-PEG-.

In certain aspects, the biocompatible polymer comprises-AKG-10diol-PEG-. The number of PEG moieties can vary depending on different situations. Higher ratios of PEG may improve hydrophilicity and biocompatibility. This can result in higher water solubility of the polymer, and can affect the ability to make microparticles.

### Polyethylene Glycol

The number of PEG moieties that is beneficial in a polymer is variable. In certain embodiments, a higher ratio of PEG improves hydrophilicity and biocompatibility. In certain embodiments, a higher ratio of PEG results in an increased water solubility of the polymer. If the polymer is too water soluble, it is not suitable to make microparticles.

### Polymeric Microparticles (MPs)

The invention also provides a polymeric microparticle (MP) composition comprising a first polymer, wherein the first polymer is the polymer of the invention.

In certain aspects, the MP composition, further comprises a second polymer.

In certain aspects, the second polymer is a polyester.

In certain aspects, the polyester is poly (L-lactic acid) (PLLA) or poly (lactic-co-glycolic acid) (PLGA).

In certain aspects, the first polymer is present at 0.1% to 99% of the composition.

In certain aspects, the first polymer is present at 1% to 50% of the composition.

In certain aspects, the first polymer is present at 1% to 10% of the composition.

In certain aspects, the first polymer is present at 1% to 5% of the composition.

In certain aspects, the MP is 50 nm to 5 μm.

In certain aspects, the MP is blended with a second polymer.

In certain aspects, the MP composition further comprises another therapeutic agent blended with the MP.

In certain aspects, the therapeutic agent is a senolytic drug.

In certain aspects, the senolytic drug is Quercetin (QT).

In certain aspects, the therapeutic agent is phenamil.

### Pharmaceutical Compositions

The invention also provides a pharmaceutical composition comprising the polymer of the invention or the MP composition of the invention, and a pharmaceutically acceptable carrier.

Biologically Active Compounds The term “biologically active compound” includes therapeutic agents that provide a therapeutically desirable effect when administered to an animal (e.g., a mammal, such as a human). Biologically active compounds that can be blended with the polymers of the invention possess at least two functional groups that can each be incorporated into an ester, thioester, or amide linkage of a polymer (as discussed in detail below), such that, upon hydrolysis of the polymer, the therapeutic agent is obtained. These groups can independently be a hydroxy group (—OH), a mercapto group (—SH), an amine group (—NHR), or a carboxylic acid (—COOH).

The biologically active compounds can also comprise other functional groups (including hydroxy groups, mercapto groups, amine groups, and carboxylic acids, as well as others) that can be used to modify the properties of the polymer (e.g. for branching, for cross linking, for appending other molecules (e.g. another biologically active compound) to the polymer, for changing the solubility of the polymer, or for effecting the biodistribution of the polymer). Lists of therapeutic agents can be found, for example, in: Physicians' Desk Reference, 55 ed., 2001, Medical Economics Company, Inc., Montvale, New Jersey; USPN Dictionary of USAN and International Drug Names, 2000, The United States Pharmacopeial Convention, Inc., Rockville, Maryland; and The Merck Index, 12 ed., 1996, Merck & Co., Inc., Whitehouse Station, New Jersey. One skilled in the art can readily select therapeutic agents that possess the necessary functional groups for incorporation into the polymers of the invention from these lists.

A specific biologically active compound that can be blended with the polymers of the invention is atorvastatin; enalapril; ranitidine; ciprofloxacin; pravastatin; clarithromycin; cyclosporin; famotidine; leuprolide; acyclovir; paclitaxel; azithromycin; lamivudine; budesonide; albuterol; indinavir; metformin; alendronate; nizatidine; zidovudine; carboplatin; metoprolol; amoxicillin; diclofenac; lisinopril; ceftriaxone; captopril; salmeterol; xinafoate; imipenem; cilastatin; benazepril; cefaclor; ceftazidime; morphine; dopamine; bialamicol; fluvastatin; phenamidine; podophyllinic acid 2-ethylhydrazine; acriflavine; chloroazodin; arsphenamine; amicarbilide; aminoquinuride; quinapril; oxymorphone; buprenorphine; butorphanol; nalbuphine. streptozocin; doxorubicin; daunorubicin; plicamycin; idarubicin; mitomycin C; pentostatin; mitoxantrone; cytarabine; fludarabine phosphate; floxuridine; cladribine; 6-mercaptopurine; thioguanine; capecitabine; docetaxel; etoposide; gemcitabine; topotecan; vinorelbine; vincristine; vinblastine; teniposide; melphalan; methotrexate; 2-p-sulfanilyanilinoethanol; 4,4′-sulfinyldianiline; 4-sulfanilamidosalicylic acid; acediasulfone; acetosulfone; amikacin; amphotericin B; ampicillin; apalcillin; apicycline; apramycin; arbekacin; aspoxicillin; azidamfenicol; aztreonam; bacitracin; bambermycin(s); biapenem; brodimoprim; butirosin; capreomycin; carbenicillin; carbomycin; carumonam; cefadroxil; cefamandole; cefatrizine; cefbuperazone; cefclidin; cefdinir; cefditoren; cefepime; cefetamet; cefixime; cefmenoxime; cefminox; cefodizime; cefonicid; cefoperazone; ceforanide; cefotaxime; cefotetan; cefotiam; cefozopran; cefpimizole; cefpiramide; cefpirome; cefprozil; cefroxadine; cefteram; ceftibuten; cefuzonam; cephalexin; cephaloglycin; cephalosporin C; cephradine; chloramphenicol; chlortetracycline; clinafloxacin; clindamycin; clomocycline; colistin; cyclacillin; dapsone; demeclocycline; diathymosulfone; dibekacin; dihydrostreptomycin; dirithromycin; doxycycline; enoxacin; enviomycin; epicillin; erythromycin; flomoxef; fortimicin(s); gentamicin(s); glucosulfone solasulfone; gramicidin S; gramicidin(s); grepafloxacin; guamecycline; hetacillin; isepamicin; josamycin; kanamycin(s); leucomycin(s); lincomycin; lomefloxacin; lucensomycin; lymecycline; meclocycline; meropenem; methacycline; micronomicin; midecamycin(s); minocycline; moxalactam; mupirocin; nadifloxacin; natamycin; neomycin; netilmicin; norfloxacin; oleandomycin; oxytetracycline; p-sulfanilylbenzylamine; panipenem; paromomycin; pazufloxacin; penicillin N; pipacycline; pipemidic acid; polymyxin; primycin; quinacillin; ribostamycin; rifamide; rifampin; rifamycin SV; rifapentine; rifaximin; ristocetin; ritipenem; rokitamycin; rolitetracycline; rosaramycin; roxithromycin; salazosulfadimidine; sancycline; sisomicin; sparfloxacin; spectinomycin; spiramycin; streptomycin; succisulfone; sulfachrysoidine; sulfaloxic acid; sulfamidochrysoidine; sulfanilic acid; sulfoxone; teicoplanin; temafloxacin; temocillin; tetroxoprim; thiamphenicol; thiazolsulfone; thiostrepton; ticarcillin; tigemonam; tobramycin; tosufloxacin; trimethoprim; trospectomycin; trovafloxacin; tuberactinomycin; vancomycin; azaserine; candicidin(s); chlorphenesin; dermostatin(s); filipin; fungichromin; mepartricin; nystatin; oligomycin(s); perimycin A; tubercidin;6-azauridine; 6-diazo-5-oxo-L-norleucine; aclacinomycin(s); ancitabine; anthramycin; azacitadine; azaserine; bleomycin(s); carubicin; carzinophillin A; chlorozotocin; chromomycin(s); denopterin; doxifluridine; edatrexate; eflornithine; elliptinium; enocitabine; epirubicin; mannomustine; menogaril; mitobronitol; mitolactol; mopidamol; mycophenolic acid; nogalamycin; olivomycin(s); peplomycin; pirarubicin; piritrexim; prednimustine; procarbazine; pteropterin; puromycin; ranimustine; streptonigrin; thiamiprine; Tomudex® (N-[[5-[[(1,4-Dihydro-2-methyl-4-oxo-6-quinazolinyl)methyl]methylamino]-2-thienyl]carbonyl]-L-glutamic acid), trimetrexate, tubercidin, ubenimex, vindesine, zorubicin; argatroban; coumetarol; dicoumarol; ethyl biscoumacetate; ethylidene dicoumarol; iloprost; lamifiban; taprostene; tioclomarol; tirofiban; amiprilose; bucillamine; gusperimus; mycophenolic acid; procodazole; romurtide; sirolimus (rapamycin); tacrolimus; butethamine; fenalcomine; hydroxytetracaine; naepaine; orthocaine; piridocaine; salicyl alcohol; 3-amino-4-hydroxybutyric acid; aceclofenac; alminoprofen; amfenac; bromfenac; bromosaligenin; bumadizon; carprofen;diclofenac; diflunisal; ditazol; enfenamic acid; etodolac; etofenamate; fendosal; fepradinol; flufenamic acid; gentisic acid; glucamethacin; glycol salicylate; meclofenamic acid; mefenamic acid; mesalamine; niflumic acid; olsalazine; oxaceprol; S-adenosylmethionine; salicylic acid; salsalate; sulfasalazine; or tolfenamic acid.

A biologically active compound suitable for blending with the polymers of the invention is morphine, dopamine, bialamicol, or tetracycline.

A biologically active compound suitable for blending with the present invention is phenamidine, acriflavine, chloroazodin, arsphenamine, amicarbilide or aminoquinuride. Another biologically active compound that can be blended with a polymer of the invention is oxymorphone, buprenorphine, butorphanol, or nalbuphine.

Another preferred biologically active compound that can be blended with a polymer of the invention is methotrexate, doxorubicin, or daunorubicin.

Another biologically active compound that can be blended with a polymer of the invention is atorvastatin, enalapril, ranitidine, pravastatin, cyclosporin, famotidine, leuprolide, acyclovir, lamivudine, budesonide, albuterol, indinavir, metformin, alendronate, nizatidine, zidovudine, carboplatin, metoprolol, lisinpril, captopril, salmeterol, cilastatin, benazepril, cefaclor, fluvastatin, quinapril, gemcitabine or vincristine.

Another biologically active compound that can be blended wtih a polymer of the invention is a nonsteroidal anti-inflammatory drug, for example, a nonsteroidal anti-inflammatory drug as described in U.S. patent application (Ser. No. 09/732,516, filed 7 Dec. 2000), 3-amino-4-hydroxybutyric acid, aceclofenac, alminoprofen, amfenac, bromfenac, bromosaligenin, bumadizon, carprofen, diclofenac, diflunisal, ditazol, enfenamic acid, etodolac, etofenamate, fendosal, fepradinol, flufenamic acid, gentisic acid, glucamethacin, glycol salicylate, meclofenamic acid, mefenamic acid, mesalamine, niflumic acid, olsalazine, oxaceprol, S-adenosylmethionine, salicylic acid, salsalate, sulfasalazine, tolfenamic acid and the like.

Another biologically active compound that can be blended with a polymer of the invention is an anti-bacterial, for example, 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefmenoxime, cefminox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin, vancomycin and the like.

Another biologically active compound that can be blended with a polymer of the invention is an anti-fungal, for example, azaserine, candicidin(s), chlorphenesin, dermostatin(s), filipin, fungichromin, mepartricin, nystatin, oligomycin(s), perimycin A, tubercidin and the like.

Another biologically active compound that can be blended with a polymer of the invention is an anti-cancer (e.g., carcinomas, sarcomas, leukemias and cancers derived from cells of the nervous system), including anti-neoplastic, for example, 6-azauridine, 6-diazo-5-oxo-L-norleucine, 6-mercaptopurine, aclacinomycin(s), ancitabine, anthramycin, azacitadine, azaserine, bleomycin(s), capecitabine, carubicin, carzinophillin A, chlorozotocin, chromomycin(s), cladribine, cytarabine, daunorubicin, denopterin, docetaxel, doxifluridine, doxorubicin, edatrexate, eflornithine, elliptinium, enocitabine, epirubicin, etoposide, floxuridine, fludarabine, gemcitabine, idarubicin, mannomustine, melphalan, menogaril, methotrexate, mitobronitol, mitolactol, mitomycin C, mitoxantrone, mopidamol, mycophenolic acid, nogalamycin, olivomycin(s), paclitaxel, pentostatin, peplomycin, pirarubicin, piritrexim, plicamycin, podophyllinic acid 2-ethylhydrazine, prednimustine, procarbazine, pteropterin, puromycin, ranimustine, streptonigrin, streptozocin, teniposide, thiamiprine, thioguanine, Tomudex® (N-[[5-[[(1,4-Dihydro-2-methyl-4-oxo-6-quinazolinyl)methyl]methylamino]-2-thienyl]carbonyl]-L-glutamic acid), toptecan, trimetrexate, tubercidin, ubenimex, vinblastine, vindesine, vinorelbine, zorubicin and the like.

Another biologically active compound that can be blended with a polymer of the invention is an anti-thrombotic, for example, argatroban, coumetarol, dicoumarol, ethyl biscoumacetate, ethylidene dicoumarol, iloprost, lamifiban, taprostene, tioclomarol, tirofiban and the like.

Another biologically active compound that can be blended with a polymer of the invention is an immunosuppressive, for example, 6-mercaptopurine, amiprilose, bucillamine, gusperimus, mycophenolic acid, procodazole, romurtide, sirolimus (rapamycin), tacrolimus, ubenimex and the like.

Another biologically active compound that can be blended with a polymer of the invention is a general or local anesthetic, for example, butethamine, fenalcomine, hydroxytetracaine, naepaine, orthocaine, piridocaine, salicyl alcohol and the like.

### Quercetin

Of all the known senolytic drugs, quercetin (QT), a naturally occurring antioxidant flavonoid, stands out for its exceptional safety profile as it is recognized as generally safe (GRAS) by the FDA. Furthermore, recent comparative studies utilizing preclinical models have underscored its superior efficacy in localized elimination of senescent cells and its potent ability to enhance osteogenesis and bone formation. QT's potent antioxidant capabilities (endowed by its unique molecule structure) has been confirmed through H₂O₂ (ROS) scavenging assays. QT is also valid to improve BMSCs osteogenic differentiation (ALP). Furthermore, it has been observed that QT treatment rescues H₂O₂ and LPS-induced cellular senescence in primary mouse BMSCs. Consistently, QT mitigates H₂O₂-inhibited osteoblastic differentiation. It has also been revealed that QT reduces the expression of LPS-induced inflammatory cytokines by macrophages. However, the utilization of QT is hindered by significant challenges, primarily stemming from its poor water solubility, chemical instability, and low bioavailability. QT has now been successfully incorporated into both PLGA and PLGA-pAKG (9:1) MPs, enabling sustained release (>5 weeks) in vitro. Notably, the findings indicate that PLGA-pAKG MPs outperform PLGA MPs in mitigating the deleterious effects of H₂O₂ and enhancing osteoblastic differentiation. Therefore, the novel pAKG MPs represent an efficient approach for intracellular drug delivery, enhancing the efficacy of QT.

### Formulations

The polymers of the invention can be formulated as pharmaceutical compositions and administered to a mammalian host, such as a human patient in a variety of forms adapted to the chosen route of administration, i.e., orally rectally, or parenterally, by intravenous, intramuscular, intraperitoneal, intraspinal, intracranial, topical or subcutaneous routes. For some routes of administration, the polymer can conveniently be formulated as micronized particles.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations preferably contain at least 0.1% of polymer by weight. The percentage of the compositions and preparations may, of course, be varied and may conveniently be between about 2 to about 80% of the weight and preferably 2 to about 60% of a given unit dosage form. The amount of polymer in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The polymer may also be administered intravenously, intraspinally, intracranially, or intraperitoneally by infusion or injection. Solutions of the polymer can be prepared a suitable solvent such as an alcohol, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile solutions or dispersions or sterile powders comprising the polymer containing the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the polymer in the required amount in the appropriate solvent with various of the other ingredients enumerated above, as required, followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

For topical administration, the present polymers can be applied in pure form. However, it will generally be desirable to administer them as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

### Dosages

Useful dosages of the polymers can be determined by comparing their in vitro activity, and in vivo activity of the therapeutic agent in animal models. Methods for the extrapolation of effective dosages in mice, and other animals, to humans are known to the art. Additionally, useful dosages can be determined by measuring the rate of hydrolysis for a given polymer under various physiological conditions. The amount of a polymer required for use in treatment will vary not only with the particular polymer selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

### Combination Therapies

The polymers of the invention are also useful for administering a combination of therapeutic agents to an animal. Such a combination therapy can be carried out by administering the polymer of the invention in addition to a different therapeutic agent. The polymer of the invention and the second agent can be blended together, administered together, or administered within a short period of time of each other.

Thus, the invention also provides a pharmaceutical composition comprising a polymer of the invention and a second therapeutic agent that is blended with the polymer of the invention. The polymers of the invention can also be administered in combination with other therapeutic agents that are effective to treat a given condition to provide a combination therapy. Thus, the invention also provides a method for treating a disease in a mammal comprising administering an effective amount of a combination of a polymer of the invention and another therapeutic agent. The invention also provides a pharmaceutical composition comprising a polymer of the invention, another therapeutic agent, and a pharmaceutically acceptable carrier.

### Preparation of Polymers of the Invention

Processes for preparing polymers of the invention are provided as further embodiments of the invention and are illustrated by the following procedures in which the meanings of the generic radicals are as given above unless otherwise qualified.

For example, a polymer of the invention can be prepared as follows: One equivalent mole of AKG was mixed with one equivalent mole of diol in a round-bottom flask without solvent. PEG was also considered as a diol. The round-bottom flask was connected to vacuum and heated to 120° C. in an oil bath. The solid mixture was melted upon heating and kept magnetic stirring for 36 hours. The resulting polymer was dissolved in minimal amount of methanol and precipitated in water. The precipitated polymer was collected by centrifuge at 1000 rpm in a conical tube and washed with methanol or water 3 times to remove unreacted monomers. Scheme I:

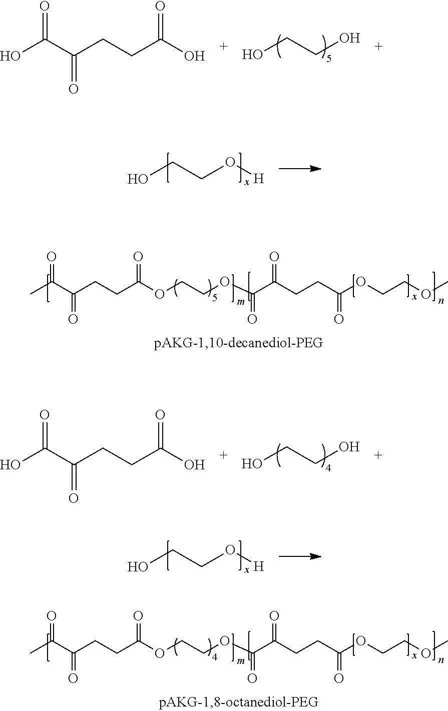

### Methods of Use

The invention also provides a method of increasing bone regeneration by administering to an animal in need of such therapy an effective amount of the polymer of the invention or the MP composition of the invention, or the pharmaceutical composition of the invention.

The invention also provides a method of increasing osteogenic differentiation by administering to an animal in need of such therapy an effective amount of the polymer of the invention or the MP composition of the invention, or the pharmaceutical composition of the invention.

The invention also provides a method of increasing intracellular phagocytosis by administering to an animal in need of such therapy an effective amount of the polymer of the invention or the MP composition of the invention, or the pharmaceutical composition of the invention.

The invention also provides a method of contacting pre-osteoblast MC3T3-E1 or primary bone marrow mesenchymal stem cells (MSCs) with the polymer of the invention or the MP composition of the invention to promote osteoblastic differentiation and/or mineralization.

In certain aspects, the cells are senescent cells.

The invention will now be illustrated by the following non-limiting Examples.

### EXAMPLE 1

Alpha-ketoglutarate (aKG or AKG) is emerging as an intriguing endogenous anti-aging molecule across various organisms. As an essential tricarboxylic acid (TCA) cycle intermediate and an energy donor, AKG was first reported to extend the lifespan of adult C. elegans by ˜50% in 2014. Furthermore, another study revealed that dietary AKG could extend Drosophila lifespan by modulating mTOR/AMPK pathways. A recent study indicated that dietary AKG can significantly expand the lifespan and healthspan of aged mice in 2020. While AKG's specific role in bone formation remains unclear, it is recognized as an essential metabolite for bone anabolism. For example, dietary AKG is known for supporting amino acid and collagen matrix synthesis. The enzyme glutaminase (GLS) and AKG production via glutamine metabolism were found essential for skeletal stem cells. Additionally, AKG production plays a crucial role in parathyroid hormone (PTH) and wingless-related integration site (Wnt)-induced bone anabolism. While circulatory AKG levels significantly decrease in older humans and mice, dietary supplementation with AKG increases circulating AKG levels in aged mice, mitigates age-related osteoporosis, and promotes new bone formation. Taken together, AKG has shown great promise in promoting bone regeneration.

The therapeutic applicability of locally applied AKG for bone regeneration is currently hindered due to poor cell permeability and lack of biomaterial-based AKG delivery. A recent investigation indicates that a cell-permeable AKG derivative (dimethyl-AKG, DMAKG) can promote osteoblastic differentiation of mouse cranial pre-osteoblastic cells and primary bone marrow mesenchymal stem cells (BMSCs). More importantly, for the first time, the in vivo study indicates that local scaffold (gelfoam)-released DMAKG significantly improves bone morphogenetic protein-2 (BMP2)-induced ectopic bone regeneration in both young and aged mice. Despite this, it is still challenging to directly apply the soluble AKG or DMAKG for tissue regeneration due to low stability in an aqueous environment and poor bioavailability.

Remarkably, akin to other metabolites like lactic acid and citric acid, alpha-ketoglutarate (AKG) can undergo polymerization, resulting in the formation of biodegradable polyesters. When these polyesters are shaped into macroparticles (MPs), they exhibit the capacity to modulate not only the immune response of dendritic cells (DCs) but also the metabolic processes and innate immune cell phenotypes (specifically macrophages and neutrophils) through phagocytosis. However, it is important to note that this phagocytosis-mediated intracellular delivery strategy may not be applicable to nonphagocytic cells (such as osteoblasts and MSCs), which have limited MPs uptake capability. Nevertheless, this implies that the AKG-based biopolymer holds promise as an innovative approach for creating biodegradable biomaterials specifically tailored for osteogenesis and bone regeneration, an area that remains largely unexplored.

Inspired by its promising reparative abilities, innovative polyesters containing AKG monomer were designed and synthesized and were used to fabricate biodegradable MPs (PAKG MPs) for bone tissue engineering applications in this study. Through tailoring the chemical composition of the co-polymers and the particle size of PAKG MPs, it was possible to significantly reduce the cytotoxicity to pre-osteoblasts and promote their osteoblastic differentiation and matrix mineralization. While MPs made by common polyesters, e.g., poly (L-lactic acid) and poly (lactic-co-glycolic acid) (PLLA & PLGA MPs) have low uptake by osteoblastic cells, the innovative PAKG MPs are highly phagocytosable for both preosteoblastic cells and BMSCs. Furthermore, blending with 1% of PAKG enables these PLLA or PLGA MPs efficient phagocytosis for intracellular drug delivery. Using the osteogenic molecule, phenamil as a model, the data indicate that PLGA-PAKG MPs mediated intracellular drug delivery can promote significantly faster and stronger osteoblastic differentiation and matrix mineralization compared to free or PLGA MPs delivered phenamil groups. The in vivo data confirm that PAKG MPs significantly improve the large bone regeneration in a mouse cranial bone defect model.

### Results

### Synthesis and Characterization of PAKG Polymers and PAKG MPs

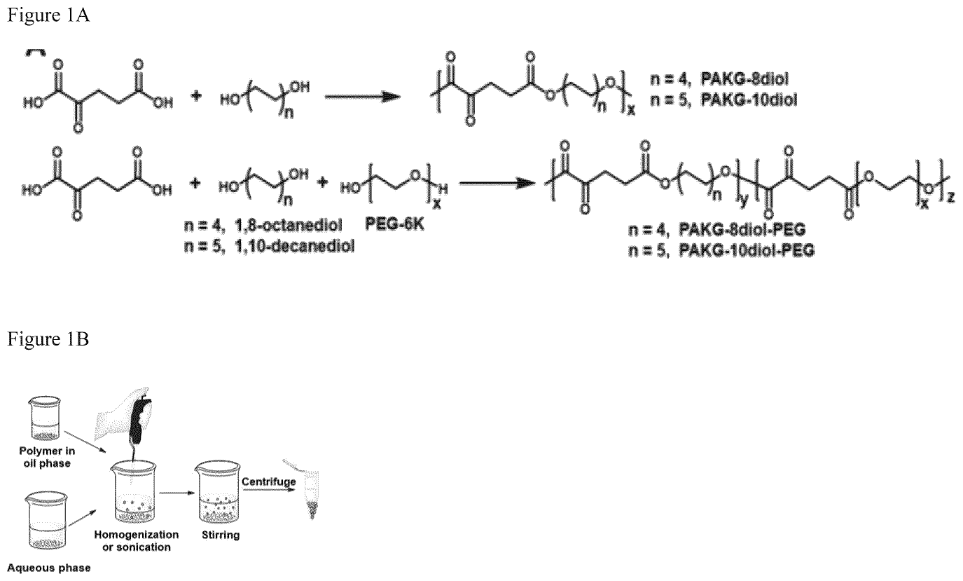

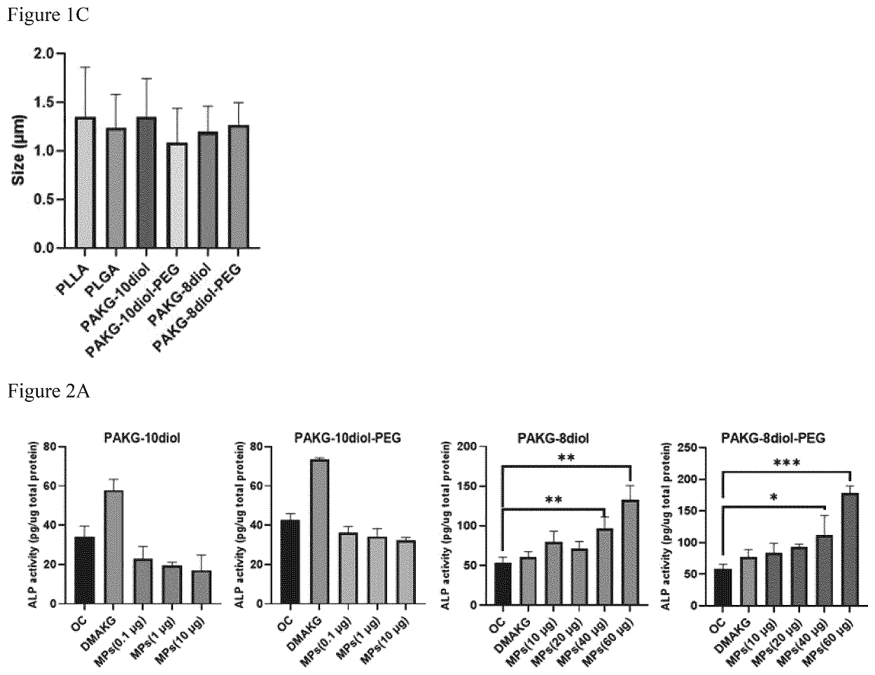

The polyesters composed of AKG and 1,8-octanediol/1,10-decanediol with or without polyethylene glycol (PEG) were synthesized through polycondensation without solvent by melting all reactants at 120° C. (FIG. 1A). The side product, water, was removed from the reaction system simultaneously by vacuum to drive the reaction towards polymer formation. The four polymers were then characterized by ¹H NMR to confirm the structure and determine the ratio of PEG in polymers. The two triplet peaks near 2.7 and 3.2 ppm corresponded to two —CH2- groups in AKG while the two triplet peaks near 4.1 and 4.3 ppm corresponded to two end —CH2- groups adjacent to O in diol for all four polymers. The three peaks between 1.2 to 1.8 ppm corresponded to the —CH2- groups between two end —CH2- groups in diol. For the two polymers containing PEG, the peak near 3.65 ppm corresponded to —CH2- groups in PEG. Therefore, according to the integration of this PEG peak, the weight ratio of PEG could be calculated to be 22% for PAKG-10diol-PEG and 13.4% for PAKG-8diol-PEG. By adjusting the ratio of starting materials, different content of PEG in resulting polymers could be achieved. PAKG-8diol-PEG with higher ratio of PEG was synthesized but was readily soluble in water, resulting in difficulty in making microparticles for the applications.

As indicated in the contact angle results, PAKG-8diol has higher hydrophilicity than PAKG-10diol due to the shorter aliphatic carbon chain of 1,8-octanediol. 1,8-octanediol is the largest aliphatic diol that is considered water soluble while 1,10-decanediol is water insoluble. The presence of PEG was another important factor that could influence the hydrophilicity of the polymers. The polymers with PEG had smaller contact angle which indicated higher hydrophilicity. Although PAKG-8diol showed higher hydrophilicity than PAKG-10diol, PAKG-10diol-PEG exhibited higher hydrophilicity than PAKG-8diol-PEG due to higher PEG ratio in the polymer chain. PAKG MPs were then fabricated using an oil-in-water emulsion method, which is illustrated in FIG. 1B. By altering the energy input method, i.e., homogenization or sonication, MPs size could be controlled precisely. After fabrication, MPs sizes and morphology were characterized using SEM. The SEM images showed that all MPs fabricated using homogenizer were spherical and had the size around 1 μm and there was no significant difference between polymers, as shown in FIG. 1C.

### Effect of PAKG MPs on Osteoblastic Differentiation and Mineralization

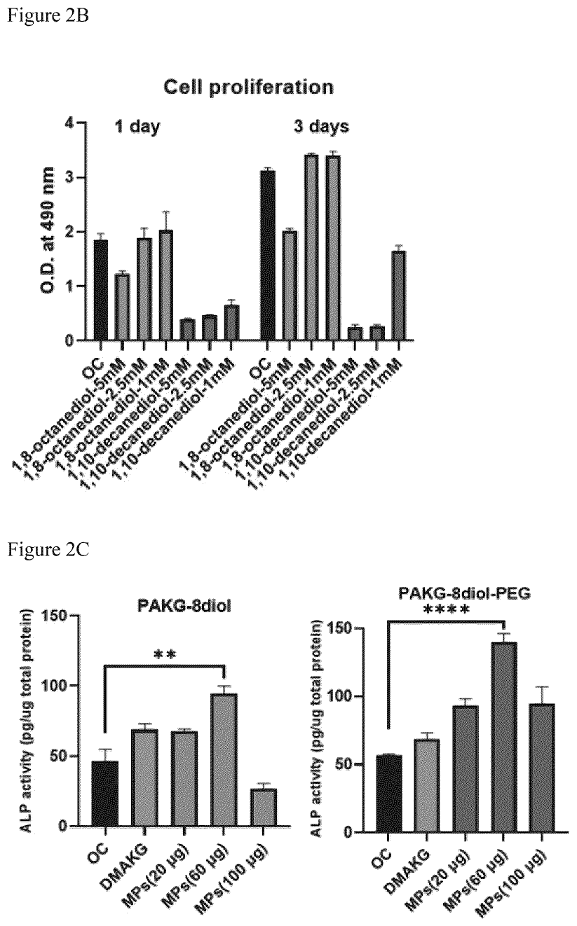

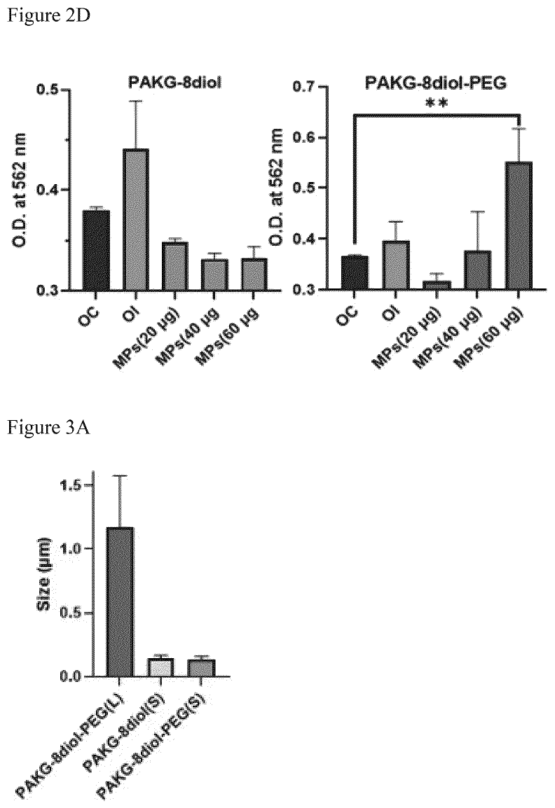

To determine the effect on osteoblast differentiation, MC3T3-E1 cells were treated with different doses of PAKG-10diol MPs, and DMAKG was used as a positive control. However, PAKG-10diol MPs strongly inhibited the ALP activity in a dose dependent manner (FIG. 2A), suggesting the potential cytotoxicity of PAKG-10diol MPs. PAKG-10diol-PEG MPs showed less inhibition of ALP but failed to improve it when a PEG moiety was introduced (FIG. 2A). Alternatively, the more hydrophilic 1,8-octanediol compared to 1,10-decanediol was tested. Both PAKG-8diol and PAKG-8diol-PEG could significantly increase ALP activity in a dose-dependent manner with up to 60 μg of MPs per well. In addition, even the lowest dose (10 μg) showed higher promotion of ALP activity compared to DMAKG (FIG. 2A).

To further characterize the biocompatibility of these PAKG polymers, the effects were studied of 1,8-octanediol and 1,10-decanediol, which are the degradation products of PAKG, on the cell proliferation of MC3T3-E1 cells. The result showed that 1,8-octanediol had little negative effect on cell proliferation up to 2.5 mM while 1,10-decanediol significantly decreased cell proliferation from 1 mM (FIG. 2B). This explained the better biocompatibility of PAKGs containing 1,8-octanediol compared to 1,10-decanediol. It was noted that neither 1,8-octanediol nor 1,10-decanediol (0.5 mM) had a detectable positive effect on ALP activity in MC3T3-E1 cells. This suggests that AKG is the main component contributing to the pro-osteogenic capability in the PAKG polymers. The addition of PEG to the PAKG MPs (PAKG-8diol-PEG MPs) further improved both the ALP activity (FIG. 2C) and mineralization (FIG. 2D) of MC3T3-E1 cells when both of them were treated at 60 μg per well. The pro-osteoblastic ability of PAKG-8diol and PAKG-8diol-PEG MPs was further confirmed by gene expression of multiple osteogenic markers (ALP, OCN, BSP, RUNX2) in MC3T3-E1. Based on these data, PAKG-8diol-PEG was determined to be the best formulation of the four PAKG polymers to make MPs for osteoblastic differentiation and used for the following experiments.

### Effect of PAKG MPs Size on Osteoblastic Differentiation and Mineralization

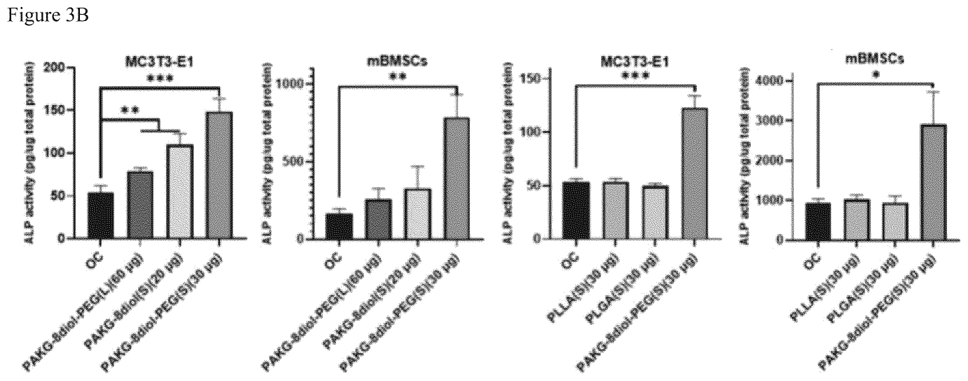

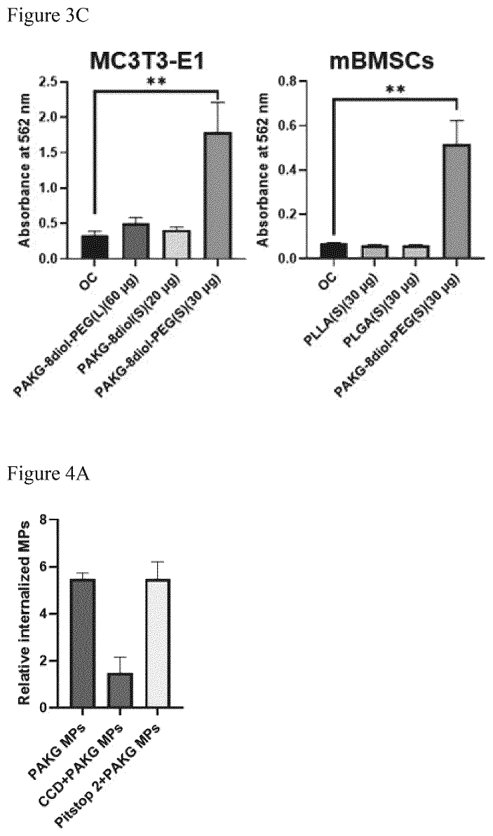

Besides improving hydrophilicity, smaller size is also known to accelerate the degradation of the polymeric MPs in part due to the larger surface area. Therefore, smaller PAKG-8diol(S) and PAKG-8diol-PEG(S) MPs were fabricated to access their bioactivities. The comparison of larger and smaller MPs is presented in FIG. 3A. The size of the smaller MPs was only about 1/10 of the size of the larger MPs. It was found that the optimal dose of PAKG-8diol(S) MPs was 20 μg and PAKG-8diol-PEG(S) was 30 μg. Then the optimal doses of PAKG-8diol(S) and PAKG-8diol-PEG(S) were compared with the optimal dose of PAKG-8diol-PEG(L) (60 μg). The ALP activity demonstrated that smaller MPs had significantly better performance in promoting osteoblastic differentiation in both MC3T3-E1 and primary mBMSCs (FIG. 3B). The pro-osteoblastic ability of PAKG-8diol-PEG(S) MPs was further confirmed by gene expressions of multiple osteogenic markers (OCN, BSP) and mineralization (FIG. 3C) using the MC3T3-E1 cells.

For comparison, PLLA(S) and PLGA(S) MPs were fabricated with similar size to PAKG(S) MPs to study their effects on the osteoblastic differentiation of MC3T3-E1 and mBMSCs. The results indicated that neither PLLA(S) nor PLGA(S) MPs could improve the ALP activity of the tested cells. Consistently, matrix mineralization data further confirmed that small PAKG-8diol-PEG(S) MPs not the PLLA or PLGA MPs could promote osteogenic differentiation of mBMSCs (FIG. 3C). Therefore, the data suggest that the distinct chemical and physical properties of the innovative PAKG MPs could significantly enhance osteoblastic differentiation and matrix mineralization.

### Osteogenesis-Related Signaling Pathways Modulated by PAKG MPs

To further characterize the pro-osteoblastic capability of the novel PAKG-8diol-PEG(S) MPs and to elucidate the underlying mechanisms, RNA-Sequencing and bioinformatics analysis using MC3T3-E1 cells was completed. A total of 217 genes were upregulated, and 137 genes were downregulated by PAKG MPs treatment compared to the nontreatment group. Gene ontology (GO) enrichment analysis indicated that the terms innate immune response, immune system process, ossification, regulation of bone mineralization, extracellular matrix, structural constituent of bone, and bone development were significantly upregulated in the PAKG MPs treatment group. Furthermore, KEEG enrichment analysis of differentially expressed genes revealed the top signaling pathways modulated by PAKG MPs are Wnt and PI3K-Akt signaling pathways among others. More specifically, Wnt4, Wnt10b, Lrp5, Fzd5, Tef7 of the Wnt signaling pathway, were consistently and significantly upregulated by PAKG MPs as determined by a Heatmap. The upregulated genes of PI3K-Akt pathways were Fgfr3, Cola2a1, and Itga10. Notably, Il1rl1, Isg15, Osmr, Ifit1, and Il18rap involved in innate immune response and inflammation were all significantly downregulated by PAKG MPs treatment. Consistent with earlier differentiation data, the osteogenic differentiation markers, e.g., Bglap and Alpl were also significantly enhanced in the PAKG group. Therefore, the RNA-seq data indicate that PAKG MPs strongly modulate Wnt, PI3K-Akt, and immune-related pathways, which are closely involved in osteogenesis.

### Osteoblastic Cells Internalization of PAKG MPs Mainly Through Phagocytosis

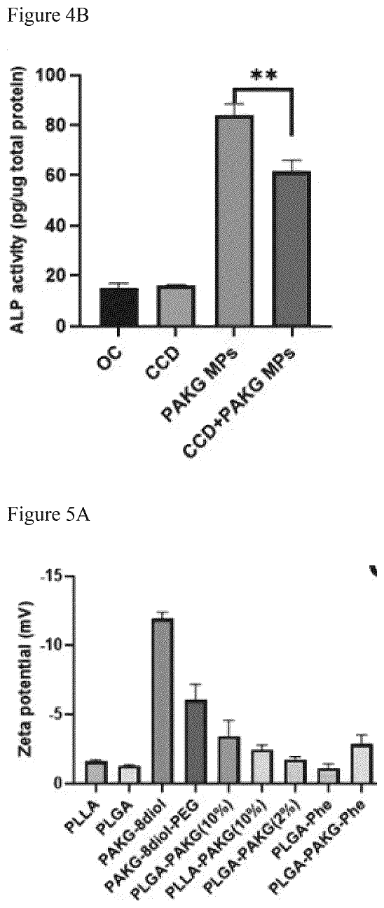

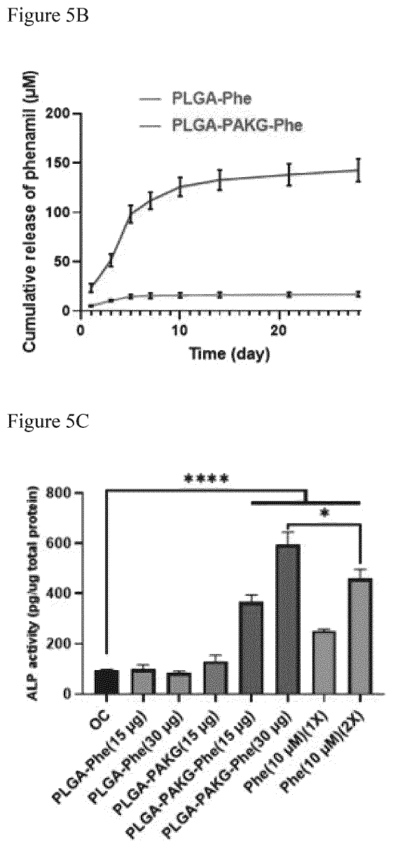

The data indicate that PLLA and PLGA MPs were scarcely internalized by osteoblastic cells while PAKG MPs (both PAKG-8diol and PAKG-8diol-PEG) with similar size could be easily phagocytosed. Cytochalasin D (CCD) is a phagocytosis inhibitor and Pitstop 2 is a clathrin-independent endocytosis inhibitor, which were used to determine the mechanism of PAKG MP uptake by osteoblastic cells. As shown in FIG. 4A the data demonstrated that CCD could significantly inhibit the internalization of PAKG MPs by MC3T3-E1 cells while Pitstop 2 had limited impact (FIG. 4A). PAKG MPs improved ALP activity of MC3T3-E1 cells were consistently reduced by CCD treatment (FIG. 4B) suggesting that MC3T3-E1 cells take PAKG MPs mainly through phagocytosis instead of receptor-mediated endocytosis.

### PAKG Enables PLGA MPs for Intra Cellular Drug Delivery

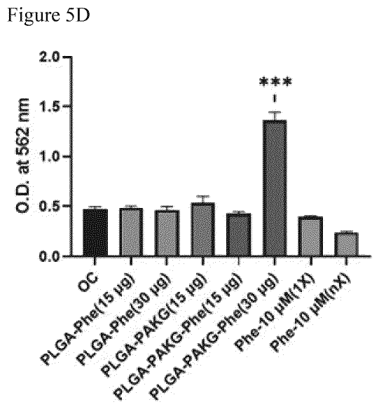

US Food and Drug Administration (FDA)-proved polymers, PLLA and PLGA are biocompatible, biodegradable, and have been widely used in drug delivery for both clinical and basic research. The data demonstrated, PLLA or PLGA MPs alone had little uptake by non-phagocytotic cells, e.g., osteoblasts and MSCs. This lack of uptake could significantly limit their applications where intracellular drug delivery is desired. For example, small molecule drug, phenamil, can promote strong osteogenic differentiation by activating intracellular BMP signaling which required the drug carriers (e.g., MPs) with efficient internalization ability by the targeting cells (e.g., osteoblasts or stem cells). Interestingly, blending with 1% of PAKG enabled the PLGA MPs efficient phagocytosis of the PLGA-PAKG MPs. A series of PLGA-PAKG MPs with different PAKG ratios (10%, 30%, 50%) were fabricated to test their effect on osteoblastic differentiation of MC3T3-E1 cells. Interestingly, ALP activities of MC3T3-E increased with the higher ratio of PAKG in the MPs. 10% PAKG in PLGA and phenamil were selected to study the effect of intracellular drug delivery of phenamil on osteoblastic differentiation of MC3T3-E1 cells. First, it was confirmed that the phagocytosis and surface property of the MPs did not change after phenamil loading. The PLGA+/−Phe MPs had little uptake by the cells while many PLGA-PAKG+/−Phe MPs were found intracellularly. The Zeta-potentials of PLGA-Phe and PLGA-PAKG-Phe MPs were close to those of PLGA and PLGA-PAKG MPs, respectively (FIG. 5A). The release profile demonstrated phenamil released from PLGA-PAKG-Phe MPs was significantly faster and greater than that from PLGA-Phe MPs (FIG. 5B). The subsequent ALP activity result of MC3T3-E1 indicated that one-dose of PLGA-PAKG-Phe MPs (30 μg) treatment group significantly promoted osteoblastic differentiation compared to one-dose PLGA-Phe MPs and onedose or multi-dose free phenamil treatment groups (FIG. 5C). Notably, one-dose of PLGA-PAKG-Phe MPs (30 μg) accelerated the mineralization process (in less than 2 weeks) compared to other treatments (FIG. 5D).

PAKG MPs promote Mouse Cranial Bone Regeneration

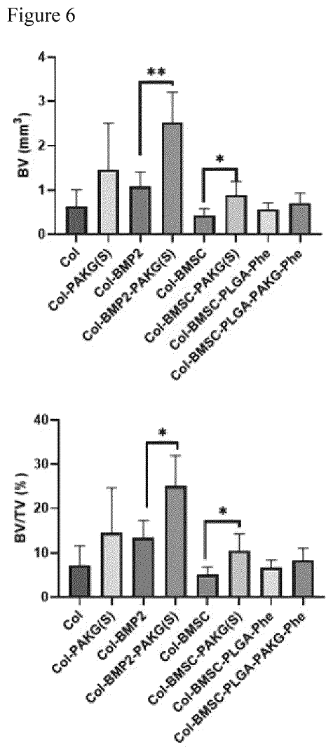

A mouse cranial bone defect model was used to investigate if PAKG MPs can induce bone regeneration in vivo. The first aim was to test if PAKG MPs can contribute to bone regeneration without exogenous cell transplantation. The uCT data, demonstrated the novel PAKG(S) MPs (Col-PAKG(S)) alone improved cranial bone regeneration compared to scaffold only (Col) (FIG. 6). In addition, low dose of BMP2 (0.5 μg per scaffold) had marginal bone regenerative capacity compared to the Col group. Excitingly, the combination group (Col-BMP2+PAKG(S)) had significantly more bone regeneration compared to the Col-BMP2 group (FIG. 6). The second aim was to study if the PAKG MPs-treated BMSCs contributed to the new bone formation in vivo. Therefore, the primary mouse BMSCs were isolated from the adult inbred male C57 BL6/J mice and treated with either the pro-osteogenic PAKG (S) MPs, the PLGA-Phe or the PLGA-PAKG-Phe MPs. These BMSCs with different treatments were seeded into collagen scaffolds and then transplanted to the mouse cranial defects. As expected, the BMSCs treated with PAKG(S) MPs (Col-BMSC-PAKG(S)) had significantly more bone formation compared to the BMSCs without MPs (Col-BMSC). Surprisingly, neither the PLGA-Phe nor PLGA-PAKG-Phe could significantly improve bone formation compared to the Col-BMSC group. Moreover, BMSCs did not show any improvement for new bone formation compared to the collagen scaffold only group (Col) (FIG. 6). H&E staining indicated obvious bone formation in the center of the defects from all the Col-PAKG(S), Col-BMP2, and Col-BMP2+PAKG(S) groups while the bone formation was limited to the edge of the defects for the collagen scaffold only group. Consistent with the uCT data, the histologic staining indicated that only the Col-BMSCPAKG(S) group had bone formation in the center of the defects while other groups with BMSCs had very limited bone formation.

### Discussion

AKG is an intriguing anti-aging metabolite, however, AKGs therapeutic potential for bone tissue engineering is unknown. the PAKG-8diol and PAKG-8diol-PEG are the first AKG based biodegradable polymers with robust pro-osteogenic abilities. Selection of diol and variation of PEG ratio may contribute to the biocompatibility and degradation rate. Furthermore, smaller MPs have a larger surface area to weight ratio, leading to a higher degradation rate and faster AKG release. The cellular interaction with MPs may also change due to size differences. Therefore, both chemical and physical features of the AKG-based polymeric MPs can be tailored for tissue regeneration and drug delivery.

The innovative PAKG MPs are more effective at promoting both pre-osteoblasts and primary BMSCs osteogenic differentiation and mineralization compared to the soluble AKG or DMAKG. This can be explained by the sustained release of bioactive AKG from the solid MPs for the targeted cells which is known crucial for drug efficacy. To better understand the pro-osteogenic capability of the PAKG MPs, comprehensive RNA-Seq analysis using the pre-osteoblast cell line MC3T3-E1 instead of heterologous primary cells, e.g., BMSCs to have better comparable information. The highly elevated osteogenic marker genes, e.g., Alpl (ALP) and Bglap (OCN) from the RNA-Seq confirmed the proosteogenic capability of PAKG MPs. Importantly, the KEGG pathway enrichment data suggests Wnt signaling pathway is the most likely target among other pathways since several key players of Wnt pathway were significantly upregulated, e.g., Wnt4, Wnt10b, Lrp5, Fzd5, and Tcf7. This is not surprising because Wnt signaling pathway is well-known for its essential anabolic roles in osteogenesis and bone regeneration. Among the upregulated genes, Wnt4 is particularly very interesting because it is produced from osteoblast and can prevent bone loss and inflammation by inhibiting NF-κB in aging mice. This is also consistent with other immune/inflammation related genes were downregulated as the data indicated. Therefore, the osteogenic activity mediated by PAKG MPs is likely to be coupled with its anti-inflammatory property to further promote bone regeneration.

Furthermore, the data identified another top target is PI3K-Akt pathway by PAKG MPs. It is interesting to note that PI3K/AKT pathway can promote bone regeneration through its crosstalk with Wnt/β-catenin pathway. Wnt signaling is known to promote bone anabolism by increasing glutamine catabolismthrough producing AKG from the TCA cycle to activate mTORCI in a in a PI3K-Akt dependent manner. However, it has not been shown if increased exogenous AKG could reversely improve Wnt and PI3K/AKT signaling. Glutamine metabolism derived AKG is reported to support amino acid biosynthesis, proliferation and osteogenic differentiation in skeletal stem cells while inhibit their adipogenic differentiation. Therefore, the data suggest that PAKG MPs produced exogenous AKG which, in-turn, promoted osteogenic differentiation through activating Wnt and PI3K/AKT signaling.

In addition to the pro-osteogenic ability on osteoblasts, it has been reported that AKG and DMAKG can significantly modulate inflammation in macrophages. The in vitro study showed that both PAKG-8diol and PAKG-8diol-PEG MPs had limited impact on LPS-induced inflammatory cytokines production by macrophages while PAKG-10diol MPs significantly and dose-dependently increased their expression. In addition, previous reports demonstrate polymer nano/microparticles are potent inflammatory stimulus for macrophages. Additionally, the cytotoxicity of 1,10-decanediol is significantly higher than 1,8-octanediol as indicated above. These factors might contribute to the pro-inflammatory effect of PAKG-10diol MPs. Although AKG is also released from the gradual degradation of PAKG MPs, the concentration of AKG might be not high enough to mitigate the immense inflammatory response induced by LPS. Moreover, the PAKG MPs demonstrated strong hydrogen peroxide (H₂O₂) scavenging capacity while neither PLGA nor PLLA MPs had any functions. Consistently, the H₂O₂-inhibited osteoblastic differentiation can be significantly rescued by PAKG MPs because AKG is a strong antioxidant and protects cells from the detrimental ROS.

One intriguing feature of PAKG MPs is their ability for efficient intracellular drug delivery through phagocytosis. It is known that large particles (>0.5 μm), such as microorganisms, can be phagocytosed by phagocytic cells, i.e., macrophages, neutrophils, and dendritic cells. The data indicated that the majority of PLLA(L) or PLGA(L) MPs stayed away from the cells, suggesting the repulsion between the MPs and the cells. The repulsion could be electrostatic repulsion from the surface charge of the cells and MPs (zeta-potential). The cell membrane surface charge is usually negative. The zetapotential results showed that PAKG-8diol(L) and PAKG-8diol-PEG(L) MPs had even stronger negative charge than PLLA (L) and PLGA (G) MPs. Another possible factor is the hydrophilicity of the polymer although PAKG-8diol has similar hydrophilicity with PLLA and PLGA. Therefore, neither surface charge nor hydrophilicity can explain why PAKG MPs can be effectively phagocytosed by osteoblasts. Excitingly, the study indicates that a small portion of PAKG can enable efficient phagocytosis of PLLA or PLGA MPs for intracellular drug delivery. This ability of PAKG has many potential important applications since both PLLA and PLGA have been widely used with established safety profiles and versatile processability.

Phenamil has shown pro-osteogenic capabilities in recent studies. However, its application has been limited by its poor aqueous solubility and low bioavailability. In other words, lower concentration of phenamil has limited pro-osteogenic capability and higher concentration causes cell death due to the high toxicity. The present results reveal that PLGA-PAKG-MPs-mediated intracellular delivery of phenamil significantly promoted osteoblastic differentiation and accelerated matrix mineralization, compared to PLGA-MPs-mediated extracellular delivery of phenarnil or free phenarnil. As shown in the release profile, blending 10% of a more hydrophilic PAKG in PLGA significantly accelerates degradation and thus increases the release rate of phenamil. Consequently, the phenamil is released intracellularly by PLGA-PAKG MPs in a relatively high concentration and in a sustainable way for over 10 days. In contrast, PLGA-MPs-mediated extracellular delivery of phenamil is too low to stimulate osteoblastic differentiation. Free phenamil at its optimal dose (10 μM from previous studies) in the culture medium is refreshed when changing culture medium. Therefore, PLGA-PAKG-Phe group has the highest ALP activity and fastest matrix mineralization.

As expected, the in vivo data indicated that PAKG MPs significantly improved cranial bone regeneration especially when they combined with the low dose BMP2. The PAKG MPs alone group showed the trend to improve bone formation although there was no statistical difference compared to control group. This could be the relative low sample number (n=3) due to the unexpected sample loss. Unexpectedly, transplanted BMSCs did not improve the overall bone formation compared to the groups without cells. Similarly, the improvement was not n by the phenamil loaded PLGA or PLGA-PAKG MPs as in vitro studies. The primary BMSCs were used from the inbred C57BL/6J mice for the in vivo implantation studies because they were considered as autologous cells with same gene background. However, some studies reported that autologous BMSCs did not generate bone in C57BL/6 mice because the recipient T cells-mediated strong inflammatory response by producing interferon gamma, tumor necrosis factor alpha, or transforming growth factor beta. This might possibly explain why all the BMSCs-transplanted group did not contribute to improved bone formation.

### Conclusion

In summary, the novel PAKG MPs composed of AKG, diol and PEG (PAKG-8diol-PEG) that can sustained release AKG upon degradation were synthesized. The in vitro data suggests both the chemical components of the co-polymers, i.e., carbon chain length of the diol and hydrophilicity, and the size of the MPs can significantly affect their cytotoxicity and pro-osteogenic activity. The RNA-Seq data suggest that PAKG MPs produced exogenous AKG promotes osteogenic differentiation through mainly activating Wnt and PI3K/AKT signaling. PAKG MPs were internalized by osteoblasts mainly through phagocytosis, while PLLA or PLGA MPs with similar size could be barely internalized. Blending PAKG with PLGA could enable efficient phagocytosis for efficient intracellular delivery. Consistent with the in vitro data, the in vivo data confirmed that PAKG MPs significantly improved the large bone regeneration in a mouse cranial bone defect model. Thus, the novel PAKG-based MPs showed great promise to improve osteogenic differentiation, bone regeneration, and enable efficient intracellular drug delivery through phagocytosis for broad regenerative medicine.

### EXPERIMENTAL SECTION

### Materials

α-Ketoglutaric acid, 1,10-decanediol, 1,8-octanediol, poly (ethylene glycol) (6,000), were purchased from Sigma (St. Louis MO, USA). Phenamil was purchased from Cayman Chemical (Ann Arbor MI, USA). Poly(L-lactide) ester terminated (1.06dL/g) and 50:50 poly (DL-lactide-co-glycolide) (0.39 dL/g) were purchased from DURECT Corporation (Birmingham AL, USA). Other chemical reagents were of analytical grade. The homogenizer was IKA dispenser (T 25 digital ULTRA TURRAX) with an 18 G tip. Sonicator was Branson Sonifier 450 with a ¼″ tip.

### Synthesis of PAKG-10Diol and PAKG-8Diol

Different PAKG polymers were synthesized using the same method. In brief, 1 equivalent mole of AKG was mixed with 1 equivalent mole of diol in a round-bottom flask without solvent. PEG was also considered as a diol. The round-bottom flask was connected to vacuum and heated to 120° C. in an oil bath. The solid mixture was melted upon heating and kept magnetic stirring for 36 hours. For PAKG-1,10-decanediol (PAKG-10diol) and PAKG-1,8-octanediol (PAKG-8diol), the resulting polymer was dissolved in minimal amount of dichloromethane and precipitated in methanol. The precipitated polymer was collected by centrifuge at 1000 rpm in a conical tube and washed with methanol or water 3 times to remove unreacted monomers. Finally, the polymers were dried in vacuum desiccator. The polymers were characterized with ¹H NMR spectroscopy and the molecular weight was evaluated with gel permeation chromatography.

### Synthesis of PAKG-10Diol-PEG, PAKG-8Diol-PEG

An amount of 1 g of a-AKG and 1 g PEG (6K) were mixed with 1.2 g of 1,10-decanediol or 1 g of 1,8-octanediol in a round-bottom flask without solvent. For PAKG-1,10-decanediol-PEG (PAKG-10diol-PEG) and PAKG-1,8-octanediol-PEG (PAKG-8diol-PEG), the resulting polymer was dissolved in minimal amount of methanol and precipitated in water. The round-bottom flask was connected to vacuum and heated to 120° C. in an oil bath. The solid mixture was melted upon heating and kept magnetic stirring for 36 hours. After reaction, the resulting polymer was dissolved in minimal amount of methanol and precipitated in water. The precipitated polymer was collected by centrifuge at 1000 rpm in a conical tube and washed with methanol or water 3 times to remove unreacted monomers. Finally, the polymers were dried in vacuum desiccator. The polymers were characterized with ¹H NMR spectroscopy and the molecular weight was evaluated with gel permeation chromatography.

### Contact Angle Measurement

To evaluate hydrophobicity/hydrophilicity of PAKG-10diol, PAKG-10diol-PEG, PAKG-8diol, PAKG-8diol-PEG, water contact angle measurement was conducted with Contact Angle Goniometer. In brief, 10 mg of polymer was dissolved in 0.2 mL of dichloromethane. On a glass slide, polymer solution was spread evenly with pipette and let solvent to evaporate. A thin film of polymer formed on the glass slide. Then a water droplet was injected onto the polymer film and an image of the droplet was captured.

### Large MPs Fabrication and Characterization

PAKG(L) MPs were fabricated using an oil-in water emulsification and solvent evaporation method. In brief, 50 mg of the PAKG polymers were dissolved in 1 mL of dichloromethane. The polymer solution was added to 10 mL of polyvinyl alcohol (PVA) (2%) in deionized water under homogenization at 10,000 rpm. After 30 seconds homogenization, the emulsion was poured int 50 mL of PVA (1%) solution and stirred at 300 rpm overnight to ensure complete removal of dichloromethane. The MPs were finally resuspended in deionized water and the concentrations of MPs were determined by freeze drying a specific volume of suspension and weighing the MPs. The size and morphology of the MPs were analyzed with SEM. Zeta-potential of MPs was analyzed with Zeta-sizer.

### Small MPs Fabrication and Characterization

PAKG(S) MPs were also fabricated using an oil-in water emulsification and solvent evaporation method. In brief, 50 mg of the PAKG polymers were dissolved in 1 mL of dichloromethane. The polymer solution was added to 10 mL of polyvinyl alcohol (PVA) (2%) solution under probe sonication. After 2 minutes of sonication, the emulsion was pured into 50 mL of PVA (1%) solution and stirred ate 300 rpm overnight to ensure complete removal of dichloromethane. The MPs were finally resuspended in deionized water and the concentrations of MPs were determined by freeze drying a specific volume of suspension and weighing the MPs. The size and morphology of the MPs were analyzed with SEM.

### Phenamil-Encapsulated Microparticles Fabrication

Phenamil-encapsulated MPs were fabricated with PLGA (PLGA-Phe) or 90% of PLGA and 10% of PAKG-8diol-PEG (PLGA-PAKG-Phe) using the same method mentioned above. In brief, 50 mg of PLGA or 45 mg of PLGA and 5 mg of PAKG-8diol-PEG were dissolved in 1.8 mL of DCM. Then 5 mg of phenamil in 200 μL of DMSO was added to the polymer solution. The mixture solution was added to 10 mL of polyvinyl alcohol (PVA) (2%) in deionized water under homogenization at 10,000 rpm. After 30 seconds homogenization, the emulsion was poured into 50 mL of PVA (1%) solution and stirred at 300 rpm overnight to ensure complete removal of dichloromethane. The MPs were collected by centrifuge in a conical tube and then washed with deionized water 5 times to ensure complete removal of PVA. The MPs were finally resuspended in deionized water and the concentrations of MPs were determined by freeze drying a specific volume of suspension and weighing the MPs.

### Phenamil Release Study

Phenamil release from PLGA-Phe and PLGA-PAKG-Phe MPs were studied using UV-Vis spectroscopy. Firstly, a standard curve of phenamil in PBS buffer was generated by preparation of phenamil in different concentrations (0, 1.25, 2.5, 5. 10, 20 μM). Then 4 mg of PLGA-Phe and PLGA-PAKG-Phe MPs were measured in a 1.5-mL centrifuge tube respectively and added 1 mL of PBS buffer for release test. Each sample was performed in triplicate. At each time point, the sample was centrifuged at 5000 rpm for 10 minutes and the supernatant was collected for UV-Vis spectroscopy test. Then 1 mL of fresh PBS was added to the sample tube for continuing phenamil release. The release was conducted for a total time of 4 weeks.

### Anti-Oxidation Property

AKG was reported to be capable of scavenging reactive oxygen species. To test the scavenging capability of PAKG-8diol-PEG MPs, 2 mg of PAKG-8diol-PEG MPs were incubated with hydrogen peroxide (100 μM). In comparison, 1 mg of AKG (free acid form), Na₂AKG (disodium salt form) and DMAKG were used to incubate with hydrogen peroxide. Negative control groups were hydrogen peroxide with addition of PLLA and PLGA MPs. After 30 minutes of incubation, hydrogen peroxide concentration in each group was measured with hydrogen peroxide detection kit.

### Cell Culture and Cell Seeding

Mouse calvaria-derived pre-osteoblasts (MC3T3-E1, from ATCC), mouse bone marrow mesenchymal stem cells (BMSCs) isolated from adult male C57BL/6J mice, human bone marrow mesenchymal stem cells (hMSCs, purchased from Lonza) were used in the in vitro cell experiments. These cells were cultured in growth medium (GM) in 10 cm polystyrene cell culture treated dish under a humidified atmosphere with 5% CO2 at 37° C. GM was prepared with Minimum Essential Medium Alpha (1×) (α-MEM, Gibco, Waltham, MA), supplemented with 10% fetal bovine serum (FBS), 100 μg/mL streptomycin sulfate, and 100 U/mL penicillin. For in vitro cell experiments, 10,000 cells (MC3T3-E1, BMSCs, or hMSCs) in 0.5 mL of GM were seeded in 24-well plate with triplicate in each experimental group. Cells were allowed to attach and proliferate for 2 days before drug/MP treatment.

### Cell Viability and Proliferation

Cytotoxicity of 1,8-octanediol and 1,10-decanediol on MC3T3-E1 cells were evaluated using a CellTiter 96 Aqueous One Solution Cell Proliferation Assay (MTS) according to the product instructions (Promega, Madison, WI, USA) on day 1 and day 3. Reacted medium was measured at 490 nm using a SpectraMax M2e Microplate Reader (Molecular Devices, San Jose, CA, USA).

### MP Treatment of Cells

For each group of cells on 24-well plate, a certain amount of MPs in a 2-mL centrifuge tube was added 1.5 mL of osteoconductive medium (OC medium). OC medium was prepared with GM supplemented with 50 g/mL of L-ascorbic acid and 2.5 mM of B-glycerophosphate salt. The MPs suspension was sonicated in bath sonicator for 5 minutes to minimize MPs aggregation. After which, the GM in each well was replaced with the suspension. Culture medium was refreshed with OC medium every 3 to 4 days without adding additional MPs.

### Gene Expression and RNA-Seq Analysis

Quantitative gene expression analysis was conducted following established protocols. In brief, RNA was extracted and purified using the miRNeasy Mini kit (QIAGEN) following the manufacturer's protocol. Subsequently, the isolated RNA served as the template for cDNA synthesis, achieved through the iScript cDNA Synthesis Kit (Bio-Rad). Then the quantitative PCR was executed using iQ SYBR Green Supermix (Bio-Rad) and evaluated using a Bio-Rad C1000 Touch PCR thermal cycler. The osteogenic biomarkers, including osteocalcin (OCN), ALP, bone sialoprotein (BSP), and RUNX2, were employed for assessing osteoblastic differentiation in MC3T3-E1, with gene expression analysis conducted after 7 days of culture. Gene primers of OCN, ALP, BSP and RUNX2 were purchased from Bio-Rad Laboratory, Inc. while the housekeeping gene primer glyceraldehyde 3-phosphate dehydrogenase was prepared at the University of Iowa.

For RNA-Seq study, mouse MC3T3-E1 cells were treated with PAKG-8diol-PEG(S) MPs (30 ug/mL) for two days before cell harvested for total RNAs isolation. The cells without MPs treatment and cultured in the same OC medium were served as control group. Poly (A) RNA sequencing library construction and sequencing, transcripts assembly and different expression analysis were performed by LC Sciences (Houston, TX). R package DESeq2 was used to perform the mRNAs differential expression analysis between the two groups. The mRNAs with the parameter of false discovery rate (FDR, specifically Q-value) below 0.05 and absolute fold change≥2 were considered differentially expressed mRNAs.

### Confocal Microscopy

MC3T3-E1 cells were used for confocal microscopy to visualize phagocytosis of microparticles. In brief, MC3T3-E1 cells were seeded on a round glass cover slip with 15 mm in diameter which could fit in the well of 24-well plate. After cell adhesion, microparticles labeled with Rhodamine B were added. After 24 hours, cells were fixed with 10% formaldehyde for 1 hour and then washed 2-3 times with PBS to remove formaldehyde. 0.1% Triton X-100 in PBS was added to the fixed cells for 5 minutes to increase permeability and washed cells 2-3 times with PBS. The actin structure was stained with fluorescent phalloidin and nuclear was stained with DAPI. Then the cells with microparticles were visualized with Confocal Microscopy.

### Animal Study

Laboratory animals' care and use were followed by the protocols approved by the Office of the Institutional Animal Care and Use Committee (IACUC) of the University of Iowa. Inbred C57BL/6J male mice (8-9 weeks, JACKSON LABS) were used to create the large size cranial bone defect model for in vivo bone regeneration study. Briefly, one 3 mm bone defect was created within the parietal bone using a trephine bur while the underlying dura mater was kept intact. The sterile collagen foam/MPs/cells/BMP2 were directly placed on the cranial defects and the overlying tissue was closed with surgical staples. Staples were removed 7 days after surgery. The porous resorbable collagen foam (ACE Surgical Supply Co., Inc.) was cut into 4 mm×4 mm×1.5 mm cuboids. These collagen scaffolds were further crosslinked using N-(3-Dimethylaminopropyl)-N′-ethylcarbodiimide hydrochloride (EDC) for 2 hours and functionalized with hydroxyapatite as previously described to improve their mechanical properties and osteo-conductivity, respectively. A total of 40 mice were used in the animal experiments, divided into 8 groups (n=5) and treated as follows: (1) Col group: modified collagen scaffolds were implanted to the defects as the scaffold only control group. (2) Col-PAKG(S) group: collagen scaffolds loaded with 60 μg of PAKG(S) MPs were implanted to the mice. (3) Col-BMP2 group: the mice were implanted with collagen scaffolds loaded with 0.5 μg of BMP2 (rhBMP2, Peprotech, Rocky Hill, NJ, USA). (4) Col-BMP2+PAKG(S) group: the mice were implanted with collagen scaffolds loaded with 0.5 ug of BMP2 and 60 μg of PAKG(S) MPs. (5) Col-BMSC group: the sterile collagen scaffolds were loaded with two million of primary mouse BMSCs (isolated from adult male C57BL/6J mice) and cultured in OC medium for 5 days before transplantation into the mice. (6) Col-BMSC-PAKG(S) group: the collagen scaffolds were loaded with the same number of BMSCs that treated with 30 μg/mL of PAKG(S) MPs overnight and cultured in OC medium for 5 days before transplantation into the mice. (7) Col-BMSCPLGA-Phe group: the same number of BMSCs were treated with 60 μg/mL of PLGA-Phe MPs overnight and seeded on the collagen scaffolds then cultured in OC medium for 5 days before transplantation into the mice. (8) Col-BMSC-PLGA-PAKG-Phe group: Same treatment as group (7) except using PLGA-PAKG-Phe MPs instead of PLGA-Phe MPs. The samples with cranial bones were retrieved and fixed for uCT (SkyScan 1272, Bruker) and histological analysis after all the mice were euthanized at 6 weeks after surgery. Some samples were not counted for data analysis because some animals died during or after the surgery or some implants shifted out of the defects. The counted sample number for each group (from 1 to 8 group) was: 5, 3, 4, 4, 5, 5, 4, and 4, respectively.

### Statistical Analysis

All experiments were performed with triplicates in each group, unless otherwise stated. Each experiment was repeated at least once. To determine the statistical significance of the differences between groups, a two-sided/-test using GraphPad Prism 9 was utilized to analyze the means between eh control group and each experimental group. *P<0.05, **P<0.01; ***P<0.001; and ****P<0.0001 were used to identify the statistically significance between individual groups.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A biocompatible polymer comprising a backbone comprising one or more units of formula (I) in the backbone: 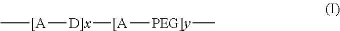
wherein
A is Alpha-ketoglutarate (AKG)
D is 1,8-octanediol (8diol) or 1,10-decanediol (10diol),
PEG is polyethylene glycol, and
x:y ratio is between 100 to 200.

2. The biocompatible polymer of claim 1, comprising-AKG-8diol-PEG-.

3. The biocompatible polymer of claim 1, comprising-AKG-10diol-PEG-.

4. A polymeric microparticle (MP) composition comprising a first polymer, wherein the first polymer is the polymer of claim 1.

5. The MP composition of claim 4, further comprising a second agent.

6. The MP composition of claim 5, wherein the second agent is a second polymer.

7. The MP composition of claim 6, wherein the second polymer is a polyester.

8. The MP composition of claim 6, wherein the polyester is poly (L-lactic acid) (PLLA) or poly (lactic-co-glycolic acid) (PLGA).

9. The MP composition of claim 4, wherein the first polymer is present at 99% of the composition.

10. The MP composition of claim 4, wherein the first polymer is present at 1% to 50% of the composition.

11. The MP composition of claim 4, wherein the first polymer is present at 1% to 10% of the composition.

12. The MP composition of claim 4, wherein the first polymer is present at 1% to 5% of the composition.

13. The MP composition of claim 4, wherein the MP is 50 nm to 5 μm.

14. The MP composition of claim 4, wherein the second agent is a biologically active compound.

15. The MP composition of claim 14, wherein the biologically active compound is a senolytic drug.

16. The MP composition of claim 15, wherein the senolytic drug is Quercetin (QT).

17. The biocompatible polymer of claim 15, wherein the biologically active compound is phenamil.

18. A pharmaceutical composition comprising a pharmaceutically acceptable carrier and
(a) the polymer of claim 1, or
(b) a MP composition comprising a first polymer, wherein the first polymer is the polymer of claim 1.

19. A method of increasing bone regeneration, increasing osteogenic differentiation, or increasing intracellular phagocytosis by administering to an animal in need of such therapy an effective amount of
(a) the polymer of claim 1, or
(b) an MP composition comprising a first polymer, wherein the first polymer is the polymer of claim 1.

20. A method of contacting pre-osteoblast MC3T3-E1 or primary bone marrow mesenchymal stem cells (MSCs) with
(a) the polymer of claim 1, or
(b) an MP composition comprising a first polymer, wherein the first polymer is the polymer of claim 1 to promote osteoblastic differentiation and/or mineralization.

21. The method of claim 20, where in the cells are senescent cells.
